(19) United States
(12) Patent Application Publication
(10) Pub. No.: US 2023/0195213 A1
(43) Pub. Date: Jun. 22, 2023

(54) CONTROL METHOD, CONTROL DEVICE, NON-TRANSITORY COMPUTER READABLE RECORDING MEDIUM, AND APPARATUS

(71) Applicant: Panasonic Intellectual Property Corporation of America, Torrance, CA (US)

(72) Inventors: Kotaro SAKATA, Osaka (JP); Tetsuji FUCHIKAMI, Osaka (JP)

(21) Appl. No.: 18/113,252

(22) Filed: Feb. 23, 2023

Related U.S. Application Data
(63) Continuation of application No. PCT/JP2021/023981, filed on Jun. 24, 2021.

(30) Foreign Application Priority Data
Sep. 2, 2020 (JP) 2020-147554

(51) Int. Cl. G06F 3/01; G06T 11/00; G06F 3/14; G16B 20/00

(52) U.S. Cl. CPC G06F 3/011; G06F 3/14; G06T 11/00; G16B 20/00

(57) ABSTRACT

A control method includes: acquiring identification information for specifying a user and gene information of the user associated with the identification information; determining a physical constitution of the user on the basis of the acquired gene information; deciding a presentation control program for controlling presentation of a content to be presented by an apparatus on the basis of the determined physical constitution; and transmitting the acquired identification information and control information for causing the apparatus to execute the decided presentation control program in association with each other.

### TECHNICAL FIELD

The present disclosure relates to a technique for controlling information presented by an apparatus.

### BACKGROUND ART

In recent years, studies have been made of a technology for presenting information suitable for an individual user to the user. For example, Patent Literature 1 discloses an information providing system that acquires user attribute information regarding user’s age, gender, occupation, hobbies, preferences, interests, consumption behavior tendency, and the like from a user attribute information recording device, selects distribution information corresponding to the user attribute information, and provides the selected distribution information to a user terminal device.

However, since the technique recited in Patent Literature 1 does not take a physical constitution of an individual user into consideration, further improvement is required in order to allow an individual user to accurately recognize a content.

### CITATION LIST

### Patent Literature

Patent Literature 1: JP 2002-351910 A

### SUMMARY OF INVENTION

An object of the present disclosure is to provide a technique enabling an individual user to accurately recognize a content.

A control method according to one aspect of the present disclosure is a control method of an apparatus that presents a content, comprising: by a computer acquiring identification information for specifying a user and gene information of the user associated with the identification information; determining a physical constitution of the user on the basis of the acquired gene information; deciding a presentation control program for controlling presentation of the content on the basis of the determined physical constitution; and transmitting, in association with each other, the acquired identification information and control information for causing the apparatus to execute the decided presentation control program.

According to the present disclosure, it is possible to cause an individual user to accurately recognize a content.

### BRIEF DESCRIPTION OF DRAWINGS

FIG. 1 is a diagram illustrating an example of an overall configuration of a control system according to a first embodiment of the present disclosure.

FIG. 2 is a block diagram illustrating an example of a configuration of a cell collection device.

FIG. 3 is a block diagram illustrating an example of a configuration of a server.

FIG. 4 is a block diagram illustrating an example of a configuration of an apparatus.

FIG. 5 is an explanatory diagram of SNP.

FIG. 6 is an explanatory diagram of a type of SNP.

FIG. 7 is a flowchart illustrating an example of processing executed when the control system according to the first embodiment of the present disclosure decides a presentation control program.

FIG. 8 is a flowchart illustrating an example of processing executed when the apparatus executes the presentation control program.

FIG. 9 is a view illustrating an example of an augmented reality screen displayed on a display device.

FIG. 10 is a view illustrating an example of the augmented reality screen displayed on the display device.

FIG. 11 is a view illustrating an augmented reality screen according to another example of the present disclosure.

FIG. 12 is a view illustrating an augmented reality screen according to still another example of the present disclosure.

FIG. 13 is a flowchart illustrating an example of processing of a control system according to a second embodiment of the present disclosure.

FIG. 14 is a flowchart illustrating an example of processing executed when an apparatus according to the second embodiment of the present disclosure executes the presentation control program.

### DESCRIPTION OF EMBODIMENTS

### How One Aspect of the Present Disclosure Has Come About

In recent years, speed-up and cost reduction of techniques for analyzing human genes are under way. Accordingly, a user can easily take a genetic testing at home or the like. The genetic testing is a test for examining a base sequence of DNA including A (adenine), T (thymine), C (cytosine), and G (guanine). The base sequence of DNA varies from a person to a person, and this difference brings about diversity of human physical constitutions. Therefore, it is possible to determine a physical constitution of a person by examining a difference in a base sequence of DNA. Then, by controlling presentation of a content in consideration of the determined physical constitution, it is possible to allow an individual user to accurately recognize the content.

However, it is not a conventional practice to determine a user’s physical constitution from gene information such as a base sequence of DNA and execute control of presentation of a content to be presented by an apparatus on the basis of the determined physical constitution. For example, in Patent Literature 1 described above, while age, gender, occupation, hobbies, preferences, interests, consumption behavior tendency, and the like of a user are taken into consideration when information is provided to the user, no physical constitution of the user is taken into consideration. Furthermore, while in Patent Literature 1, information corresponding to an attribute of a user is provided, presentation of the information is not controlled. Therefore, conventional techniques are insufficient to cause an individual user to accurately recognize a content.

Therefore, the present inventor has obtained knowledge that it is possible to cause an individual user to accurately recognize a content by controlling presentation of the content in consideration of a physical constitution of the user obtained from an analysis result of gene information of the user, and has arrived at each aspect of the present disclosure.

A control method according to one aspect of the present disclosure is a control method of an apparatus that presents a content, comprising: by a computer acquiring identification information for specifying a user and gene information of the user associated with the identification information; determining a physical constitution of the user on the basis of the acquired gene information; deciding a presentation control program for controlling presentation of the content on the basis of the determined physical constitution; and transmitting, in association with each other, the acquired identification information and control information for causing the apparatus to execute the decided presentation control program.

According to this configuration, since a physical constitution of a user is determined on the basis of the gene information, the physical constitution of the user can be accurately determined. Then, a presentation control program for controlling presentation of a content is decided the basis of the determined physical constitution, and the control information that causes the apparatus to execute the decided presentation control program and the identification information are transmitted. Therefore, the apparatus can specify a presentation control program corresponding to the identification information. This enables the apparatus to cause an individual user to accurately recognize a content.

In the control method, the physical constitution may be a physical constitution related to at least one of attentiveness and memory of a user.

According to the present configuration, since the presentation control program is selected on the basis of at least one of attentiveness and memory of an individual user, it is possible to cause the individual user to recognize a content more accurately.

In the control method, the determination may include determining whether the attentiveness is low or high on the basis of the gene information, and the presentation control program decided in a case where the attentiveness is determined to be low may be a program that changes at least one of a display mode and a display method of the content so as to make the content be conspicuous as compared with the presentation control program decided in a case where the attentiveness is determined to be high.

According to the present configuration, in a case where user’s attentiveness is determined to be low, the content is displayed in a more conspicuous display mode and/or display method than in a case where the user’s attentiveness is determined to be high. As a result, it is possible to enhance accuracy in recognizing a content for a user having low attentiveness.

In the control method, the determination may include determining whether the attentiveness is low or high, and the presentation control program decided in a case where the attentiveness is determined to be high may be a program that changes at least one of a display mode and a display method of the content so as to make the content be less conspicuous as compared with the presentation control program decided in a case where the attentiveness is determined to be low.

According to this configuration, in a case where user’s attentiveness is determined to be high, the content is displayed in a less conspicuous display mode and/or display method as compared with a case where the user’s attentiveness is determined to be low. This enables a user having high attentiveness to accurately recognize a content without feeling troublesome.

In the above control method, the determination may include determining whether the memory is bad or good, and the presentation control program decided in a case where the memory is determined to be bad may be a program that changes at least one of a display mode and a display method of the content so as to enable the user to better memorize as compared with the presentation control program decided in a case where the memory is determined to be good.

According to this configuration, in a case where user’s memory is determined to be bad, a content is displayed in a display mode and/or a display method that enables a user to better memorize than in a case where the user’s memory is determined to be good. Therefore, it is possible to cause a user having bad memory to more reliably memorize the content.

In the control method, a change of the display mode may include at least one of a change in contrast of the content and a change in size of the content.

According to the present configuration, since contrast and/or size of a content is changed, it is possible to realize display of the content suitable for a user’s physical constitution.

In the control method, a change of the display method may include at least one of a change in a display position of the content, a change in a display time of the content, and a change in the number of times of displaying the content.

According to the present configuration, since at least one of a display position of a content, a display time, and the number of times of displaying is changed, it is possible to realize display of the content suitable for a user’s physical constitution.

In the control method, the apparatus may be an augmented reality apparatus.

According to the present configuration, in an augmented reality apparatus having a restriction that a viewing angle of a user is limited, appropriate information can be accurately presented to a user according to a physical constitution of the user.

In the control method, the gene information may include information indicating a base sequence of a nucleic acid of the user.

According to this configuration, since the gene information is information indicating a base sequence of a nucleic acid of the user, a physical constitution of the user can be accurately determined.

In the control method, the determination may include detecting a single nucleotide polymorphism of the gene information, and determining the physical constitution on the basis of a detection result.

According to this configuration, since a physical constitution of a user is determined on the basis of a single nucleotide polymorphism of the gene information, the physical constitution of the user can be accurately determined.

In the control method, the control information may include the presentation control program.

According to this configuration, since a presentation control program is transmitted, it is possible to cause an apparatus to execute the presentation control program without causing the apparatus to hold the presentation control program in advance.

In the control method, the control information may include information for causing the apparatus having a plurality of presentation control programs to execute the decided presentation control program.

According to this configuration, it is possible to cause an apparatus to execute a presentation control program without transmitting the presentation control program.

An apparatus according to another aspect of the present disclosure is an apparatus that presents a content, the apparatus including: a reception part that receives control information for controlling execution of a presentation control program for controlling presentation of the content, and identification information of a user associated with the control information; the presentation control program being decided on the basis of a physical constitution of the user determined from gene information of the user, a memory that stores the control information and the identification information in association with each other; a sensor that detects a surrounding user; a specifying part that specifies identification information of the surrounding user from detection data of the sensor; and an execution part that specifies control information corresponding to the specified identification information from the memory and executes a presentation control program corresponding to the specified control information.

According to this configuration, the control information for controlling execution of a presentation control program decided on the basis of a physical constitution of a user and the identification information of the user associated with the control information are stored in the memory in association with each other. Then, the identification information of a user around the apparatus is specified from detection data of the sensor, the control information corresponding to the specified identification information is specified from the memory, and the presentation control program corresponding to the specified control information is executed. This enables an apparatus to cause a surrounding user to accurately recognize a content.

The present disclosure can be also implemented as a program for causing a computer to execute each characteristic function included in such a control method, or as a system that operates with the program. It is needless to say that such a computer program can be distributed using a computer-readable non-transitory recording medium such as a CD-ROM, or via a communication network such as the Internet.

Each of embodiments described below illustrates a specific example of the present disclosure. Numerical values, shapes, components, steps, an order of steps, and the like shown in the embodiments below are examples, and are not intended to limit the present disclosure. Further, among components in the embodiments below, a component that is not described in an independent claim representing a highest concept will be described as an optional component. In all the embodiments, respective contents can be combined.

### First Embodiment

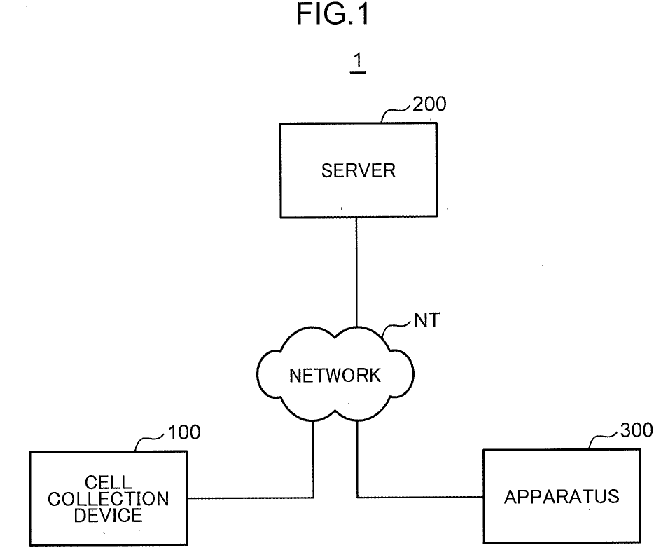

FIG. 1 is a diagram illustrating an example of an overall configuration of a control system 1 according to a first embodiment of the present disclosure. The control system 1 includes a cell collection device 100, a server 200, and an apparatus 300. The cell collection device 100, the server 200, and the apparatus 300 are communicably connected to each other via a network NT. The network NT is, for example, a public communication line such as the Internet. Note that the network NT may be a local area network. The cell collection device 100 is installed, for example, in a house of a user.

The cell collection device 100 is configured with, for example, a sequence decoding device also referred to as a DNA sequencer. The cell collection device 100 is a device that collects a cell of a user and extracts gene information of the user. The gene information includes information indicating a base sequence of DNA contained in the user’s cell. The cell collection device 100 transmits the extracted gene information to the server 200 in association with a user ID that is identification information of the user.

The server 200 is configured with a cloud server including one or more computers, for example. The server 200 determines a physical constitution of a user on the basis of the gene information transmitted from the cell collection device 100, decides a presentation control program suitable for the physical constitution, and transmits the decided presentation control program to the apparatus 300 in association with the user ID.

The apparatus 300 is configured with a device that presents a content. The apparatus 300 executes a presentation control program transmitted from the server 200. In the present embodiment, the apparatus 300 is configured with an augmented reality apparatus that displays an augmented reality image. In this case, the content includes an augmented reality image. The augmented reality apparatus is, for example, a head-up display, a head-mounted display, smart glasses, or smart contact lenses. Note that these are examples, and any apparatus may be employed as the apparatus 300 as long as the apparatus is provided with a display device that displays a content, such as a smartphone, a tablet terminal, a television, or a personal computer. The apparatus 300 may be an electric apparatus (e.g., a cooking apparatus) including a display panel. In the following description, the apparatus 300 is assumed to be a head-up display mounted on a vehicle such as an automobile.

When the apparatus 300 is mounted on a vehicle, the cell collection device 100 may be mounted on the vehicle. In addition, the cell collection device 100 may be mounted on the apparatus 300. In addition, the cell collection device 100 may be installed in an external organization that conducts genetic testing.

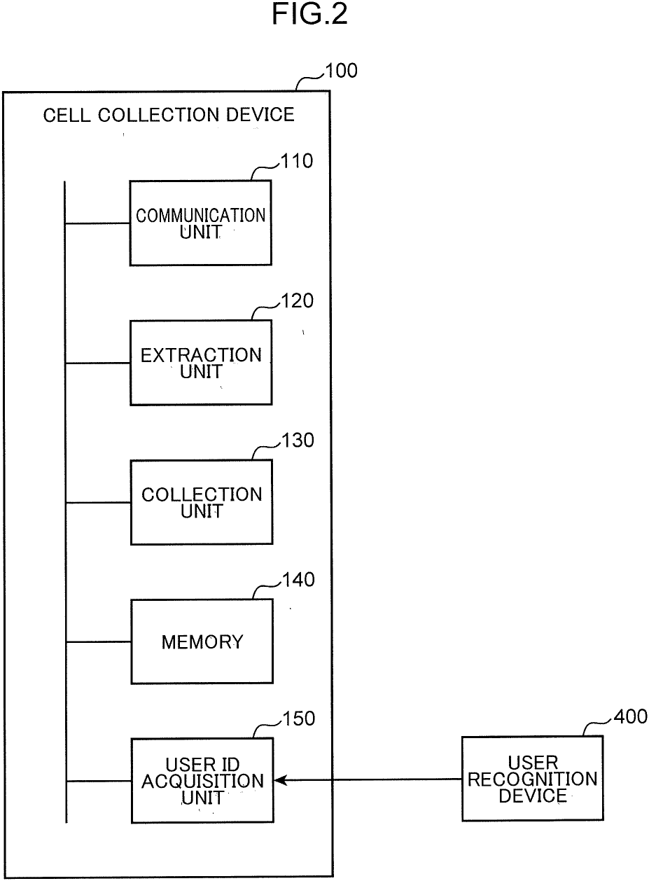

The foregoing is the entire configuration of the control system 1. Next, details of each component of the control system 1 will be described. FIG. 2 is a block diagram illustrating an example of a configuration of the cell collection device 100. The cell collection device 100 includes a communication unit 110, an extraction unit 120, a collection unit 130, a memory 140, and a user ID acquisition unit 150.

The communication unit 110 is configured with a communication circuit that connects the cell collection device 100 to the network NT. The communication unit 110 transmits gene information extracted by the extraction unit 120 to the server 200 in association with a user ID of a user who has provided the gene information.

The collection unit 130 collects a cell of a user and supplies the collected cell to the extraction unit 120.

The extraction unit 120 includes a labeling part, a light source, an image sensor, and a processor. The labeling part applies a fluorescent label for each type of base (A, T, G, C) to DNA of a cell collected by the collection unit 130. The light source irradiates DNA having an applied fluorescent label with light. The image sensor detects fluorescence emitted from DNA by light from the light source. The processor generates gene information including information indicating a base sequence of DNA on the basis of fluorescence detected by the image sensor.

The memory 140 is configured with a nonvolatile storage device that cannot be rewritten such as a flash memory. The memory 140 stores, for example, a user ID.

The user ID acquisition unit 150 includes, for example, a communication interface for communicating with a user recognition device 400, and acquires a user ID of a user who has provided gene information. Alternatively, the user ID acquisition unit 150 may acquire a user ID from memory 140. Alternatively, the user ID acquisition unit 150 may acquire a user ID input by a user using an operation device (not illustrated).

For example, when the cell collection device 100 is mounted on an electric apparatus (e.g., an electric toothbrush) used for each individual user, the memory 140 can store an user ID of a user who provides the gene information. In this case, the user ID acquisition unit 150 may acquire the user ID stored in the memory 140 as a user ID of a user who provides the gene information.

For example, the cell collection device 100 may be mounted on an operation switch of a lighting apparatus or the like which a user frequently touches. In this case, the cell collection device 100 may acquire the user ID from the user recognition device 400 provided around the operation switch.

When detecting a user touching the operation switch, the user recognition device 400 transmits a user ID of the user who has touched the operation switch to the user ID acquisition unit 150. The user recognition device 400 may detect the user who has touched the operation switch using, for example, image recognition processing. Specifically, the user recognition device 400 includes a camera, a communication unit, and a processor. The camera constantly captures an image around the operation switch. The processor executes image processing on image data captured by the camera, and detects whether or not a certain user has touched the operation switch. In a case where the processor detects a certain user touching the operation switch, the processor executes face recognition processing to determine to which user among users registered in advance, the user corresponds, and detects a user ID of the determined user as the user ID of the user who has touched the operation switch. The communication unit may input the user ID detected by the processor to the user ID acquisition unit 150.

Alternatively, the user recognition device 400 may detect the user ID by fingerprint recognition. In this case, the user recognition device 400 is configured with a fingerprint recognition device provided in the operation switch.

Alternatively, the user recognition device 400 may be mounted on the apparatus 300. In this case, the user recognition device 400 may detect a user from which a cell is collected by the cell collection device 100 in response to a user ID acquisition request output from the user ID acquisition unit 150, and input a user ID of the user to the user ID acquisition unit 150.

In a case where the cell collection device 100 is mounted on an electric toothbrush, the collection unit 130 may collect saliva of the user and collect a cell from the collected saliva. In a case where the cell collection device 100 is mounted on the operation switch, the collection unit 130 may collect sweat of a user and collect a cell of the user from the collected sweat.

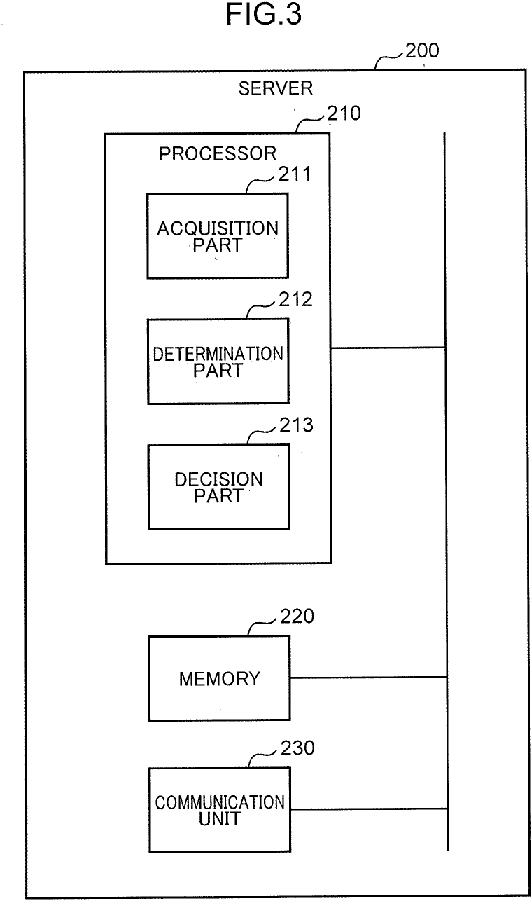

FIG. 3 is a block diagram illustrating an example of a configuration of the server 200. The server 200 includes a processor 210, a memory 220, and a communication unit 230 (an example of a transmission part). The processor 210 is configured with, for example, a CPU. The processor 210 includes an acquisition part 211, a determination part 212, and a decision part 213. The acquisition part 211 to the decision part 213 may be implemented by execution of a predetermined program by the processor 210, or may be configured with a dedicated hardware circuit.

The acquisition part 211 acquires a user ID and the gene information transmitted by the cell collection device 100 via the communication unit 230. The acquisition part 211 applies a time stamp to the acquired gene information, and stores the gene information to which the time stamp is applied in the memory 220 in association with the user ID. As a result, time-series data of the gene information for each user is accumulated in the memory 220.

The determination part 212 determines a physical constitution of a user who has provided the gene information on the basis of the gene information acquired by the acquisition part 211. Here, the determination part 212 detects a single nucleotide polymorphism (SNP) of a base sequence indicated by the gene information and a type of SNP, and determines a physical constitution of the user on the basis of a detection result. Specifically, the determination part 212 determines whether or not a SNP has appeared at a predetermined gene locus on a base sequence related to a physical constitution to be determined. In a case where a SNP has appeared at the predetermined gene locus, the determination part 212 specifies a type of the SNP. Then, the determination part 212 determines a physical constitution to be determined from the specified type. For example, the determination part 212 may determine a SNP type from a pattern of bases of SNP located at the same gene locus in homologous chromosomes.

Although base sequences of a human being are 99.9% identical, 0.1% are different. This difference causes a difference in appearance, ability, physical constitution, and the like. When a difference in base sequence appears at a frequency of 1% or more in a certain human group, the difference in the base sequence is called polymorphism. When a difference in base sequence appears at a frequency of 1% or less, the difference in base sequence is called mutation or rare variation. Polymorphism has various types, and among them, SNP is one in which one base is replaced by another base. SNP is estimated to be present at a probability of one in 500 to 1000 bases, and is estimated to be present at about ten million locations.

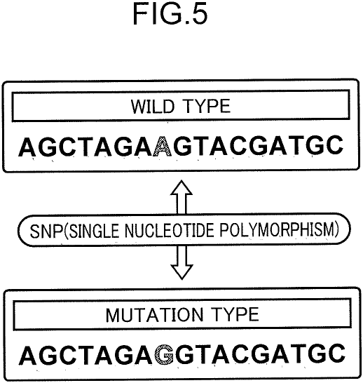

FIG. 5 is an explanatory diagram of SNP. In the example of FIG. 5, A in a normal gene sequence (wild type) is mutated to G.

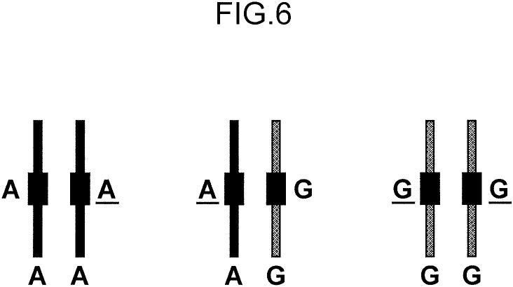

A humans being inherits one gene sequence from each of his/her father and mother, and there are three combinations. Therefore, one SNP has three types. FIG. 6 is an explanatory diagram of SNP types. For example, in the SNP mutation of A to G as shown in the example of FIG. 5, there are three SNP types, AA, AG, and GG.

For example, when SNPs of parents of a child are of the AG type, the child will inherit one A sequence and one G sequence of each of the parents. Therefore, the SNP mutation of A to G will have three types, AA, AG, and GG as shown in FIG. 6.

While there are many SNPs, it has been shown that a specific SNP is associated with a specific disease. Such SNP associated with a certain disease is referred to as “disease-related SNP”.

Examples of a disease-related SNP include a “metabolically related SNP”. “Metabolically-related SNP” is a SNP related to “metabolic syndrome”. A type of “metabolically related SNP” makes it possible to determine whether a person in question has a “physical constitution for getting fat” or a “physical constitution not for getting fat”.

Other examples of the disease-related SNP include a SNP related to an alcoholysis enzyme. A combination of GG as a SNP type related to an alcoholysis enzyme is called GG homotype. A person having this SNP type is resistant to alcohol. A combination of AG as a SNP type related to an alcoholysis enzyme is called AG heterozygous type. A person having this SNP type is naturally vulnerable to alcohol. A combination of AA as a SNP type related to an alcoholysis enzyme is called AA homotype. A person having this SNP type lacks metabolic activity for alcohol and cannot drink alcohol by nature.

As described above, when a specific SNP and a type of the SNP are known for a certain person, a physical constitution of the person can be found.

In the present embodiment, as physical constitutions, the determination part 212 determines, in particular, a physical constitution related to user’s attentiveness and a physical constitution related to user’s memory. Attentiveness represents ability to concentrate on one thing, which is a concept including concentration. Examples of genes related to attentiveness include KIBRA and SLC6A2. KIBRA is a gene that produces one type of phosphorylated protein. When the gene has a TT type and a TC type, a human being tends to have high attentiveness. Therefore, when KIBRA has the TT type and the TC type, the determination part 212 determines that attentiveness is high. On the other hand, when the type of KIBRA is other than the TT type and the TC type, the determination part 212 determines that attentiveness is low.

SLC6A2 is a gene that produces one type of protein that transports neurotransmitters, and is known to be involved in reuse of neurotransmitters (norepinephrine). When SLC6A2 has the TT type and the TC type, attentiveness tends to be low. Therefore, when SLC6A2 has the TT type and the TC type, the determination part 212 determines that attentiveness is low. On the other hand, when the type of SLC6A2 is other type than the TT type and the TC type, the determination part 212 determines that attentiveness is high.

The determination part 212 may evaluate attentiveness and concentration in a stepwise manner from determination results of KIBRA and SLC6A2, respectively. For example, in a case where both KIBRA and SLC6A2 indicate low attentiveness, the determination part 212 may set an evaluation value V1 of attentiveness to V1 = 1. In addition, the determination part 212 may set the evaluation value V1 to V1 = 2 in a case where while one of KIBRA and SLC6A2 indicates high attentiveness, the other indicates low attentiveness. Further, in a case where both KIBRA and SLC6A2 indicate high attentiveness, the determination part 212 may set the evaluation value V1 to V1 =3.

Although the determination part 212 determines memory by using all of KIBRA and SLC6A2, this is an example, and attentiveness may be determined using one of them. Further, if there is another gene involved in attentiveness, the gene may be used to determine attentiveness.

Examples of genes related to memory include KIBRA, DTNBP1, and PAH. When KIBRA has the TT type and the TC type, a human being tends to have good memory. Therefore, when KIBRA has the TT type and the TC type, the determination part 212 determines that memory is good. On the other hand, when the type of KIBRA is other than the TT type and the TC type, the determination part 212 determines that memory is bad.

DTNBP1 is a gene involved in formation of intracellular organelles. When DTNBP1 has a GG type, a human being tends to have bad memory. Therefore, when DTNBP1 has the GG type, the determination part 212 determines that memory is bad. On the other hand, when the type of DTNBP1 is other than the GG type, it is determined that memory is good.

PAH is a gene that produces phenylalanine hydroxylase. When PAH has the GG type, a human being tends to have bad memory. Therefore, when PAH has the GG type, the determination part 212 determines that memory is bad. On the other hand, when the type of PAH is other than the GG type, the determination part 212 determines that memory is good.

The determination part 212 may evaluate memory in a stepwise manner from determination results of KIBRA, DTNBP1, and PAH respectively. For example, in a case where all of KIBRA, DTNBP1, and PAH indicate bad memory, the determination part 212 may set an evaluation value V2 of memory to V2 = 1. In addition, the determination part 212 may set the evaluation value V2 to V2 = 2 in a case where any two of KIBRA, DTNBP1 and PAH indicate bad memory, and the remaining one indicates good memory. The determination part 212 may set the evaluation value V2 to V2 = 3 in a case where any one of KIBRA, DTNBP1 and PAH indicates bad memory, and the remaining two indicate good memory. The determination part 212 may set the evaluation value V2 to V2 = 4 in a case where all of KIBRA, DTNBP1 and PAH indicate good memory.

Although the determination part 212 determines memory using all of KIBRA, DTNBP1 and PAH, this is an example, and memory may be determined using any one or two of them. Further, if there is another gene involved in memory, the gene may be used to determine memory.

The decision part 213 decides one presentation control program from among a plurality of presentation control programs for controlling presentation of a content on the basis of a physical constitution determined by the determination part 212. The decision part 213 transmits the decided one presentation control program to the apparatus 300 via the communication unit 230 in association with the user ID.

In a case where the determination part 212 determines that attentiveness is low, the decision part 213 decides one presentation control program for changing at least one of a display mode and a display method of a content so as to make the content be more conspicuous than in a case where the attentiveness is determined to be high.

In other words, in a case where the determination part 212 determines that the attentiveness is high, the decision part 213 decides one presentation control program for changing at least one of the display mode and the display method of a content so as to make the content less conspicuous than in a case where the attentiveness is determined to be low.

Specifically, the decision part 213 decides one presentation control program for changing at least one of the display mode and the display method of the content so that a degree of conspicuousness becomes higher as the value of the evaluation value V1 set by the determination part 212 becomes smaller.

In addition, in a case where the determination part 212 determines that memory is bad, the decision part 213 decides one presentation control program for changing at least one of the display mode and the display method of a content so as to make the content be more conspicuous than in a case where the memory is determined to be good.

Specifically, the decision part 213 decides one presentation control program for changing at least one of the display mode and the display method of the content so that the degree of conspicuousness becomes higher as the value of the evaluation value V2 set by the determination part 212 becomes smaller.

The change of the display mode includes, for example, at least one of a change in contrast of a content and a change in size of the content. For example, a contrast of a content is set such that the contrast with respect to the background becomes higher as the evaluation value V 1 or the evaluation value V2 becomes smaller. For example, the size of the content is set to be larger as the evaluation value V1 or the evaluation value V2 becomes lower.

The change of the display mode may be, for example, a change in a length of a display character string. As described above, for example, the contrast of the content is set such that the contrast with respect to the background becomes higher as the evaluation value V1 or the evaluation value V2 becomes smaller. For example, the size of the content is set to be larger as the evaluation value V1 or the evaluation value V2 becomes lower. In addition, the length of the character string is set to be shorter as the evaluation value V1 or the evaluation value V2 becomes lower. For example, “Please be careful” is set to be short as “caution”.

The change of the display method includes, for example, at least one of a change in a display position of the content, a change in display time of the content, and a change in the number of times of displaying the content. For example, the display position of the content is changed so as to approach the center of a field of view of a user as the evaluation value V1 or the evaluation value V2 becomes lower. For example, the display time of the content is changed to be longer as the evaluation value V1 or the evaluation value V2 becomes lower. For example, the number of times of displaying the content is changed to be larger as the evaluation value V1 or the evaluation value V2 becomes lower.

For example, a content for which at least one of the display mode and the display method is changed according to attentiveness and a content for which at least one of the display mode and the display method is changed according to memory may be distinguished from each other. Note that this distinction may overlap for some content.

In a case where the apparatus 300 is an onboard apparatus such as a head-up display, the content may include, for example, a content related to a car distance between a preceding vehicle and an own vehicle, a traveling speed of the own vehicle, an engine speed, a remaining distance to a destination, and the like. Further, the content may include, for example, a content for guiding a lane change, a content for notifying a direction of a road, a content for guiding right turn or left turn during navigation to a destination, and the like. These contents are examples of augmented reality images.

The memory 220 is configured with a storage device such as a hard disk drive or a solid state drive. The memory 220 stores a plurality of presentation control programs. Here, the memory 220 stores a presentation control program and physical constitution information indicating a physical constitution in association with each other. Specifically, the memory 220 may store a plurality of presentation control programs prepared in advance according to a combination of the evaluation value V1 and the evaluation value V2, and the combination of the evaluation value V1 and the evaluation value V2 in association with each other. For example, in the above-described example in which the evaluation value V1 has three values of 1 to 3 and the evaluation value V2 has four values of 1 to 4, the number of types of presentation control programs is 12. In this case, each of 12 types of presentation control programs is stored in the memory 220 in association with a combination of the evaluation value V1 and the evaluation value V2. Note that information indicating the combination of the evaluation value V1 and the evaluation value V2 is an example of the physical constitution information.

Therefore, the decision part 213 may decide a presentation control program corresponding to a combination of the evaluation value V1 and the evaluation value V2 determined by the determination part 212 as one presentation control program.

The communication unit 230 is configured with a communication circuit that connects the server 200 to the network NT. The communication unit 230 transmits a presentation control program decided by the decision part 213 and a user ID of a user corresponding to the decided presentation control program to the apparatus 300 in association with each other.

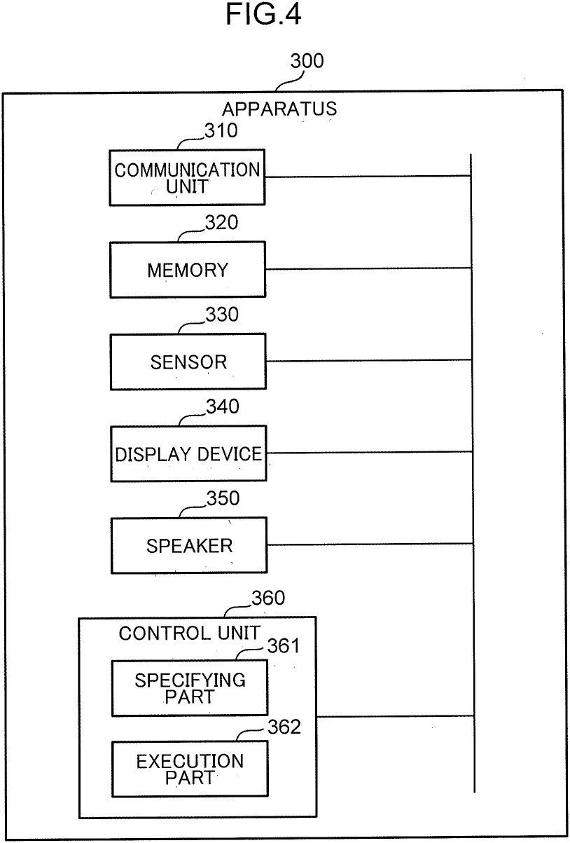

FIG. 4 is a block diagram illustrating an example of a configuration of the apparatus 300. The apparatus 300 includes a communication unit 310 (an example of a reception part), a memory 320, a sensor 330, a display device 340, a speaker 350, and a control unit 360. The communication unit 310 is configured with a communication circuit that connects the apparatus 300 to the internal network NT. The communication unit 310 receives a presentation control program and a user ID transmitted from the server 200.

The memory 320 is configured with a nonvolatile storage device that cannot be rewritten such as a flash memory. The memory 320 stores, in association with each other, a presentation control program and a user ID received from the server 200 by the communication unit 310. Further, the memory 320 stores a feature value of one or more users registered in advance and an user ID in association with each other. A feature value of a user is, for example, a feature value of a face, a feature value of a voice, or the like.

The sensor 330 is a sensor for detecting a surrounding user. Since the apparatus 300 is a head-mounted display in the present embodiment, the sensor 330 is configured with an image sensor or a microphone provided in a driver’s seat of the vehicle.

The display device 340 is configured with a projection device that projects a content onto a windshield of a vehicle. The display device 340 displays various contents as a result of execution of a presentation control program by an execution part 362. In a case where the apparatus 300 is not configured with a head-up display, the display device 340 is configured with a liquid crystal panel or an organic EL panel, or the like.

The speaker 350 outputs a content formed of various sounds as a result of execution of a presentation control program by the execution part 362. In other words, in the present embodiment, the content includes not only an image but also sound.

The control unit 360 includes a processor such as a CPU. The control unit 360 includes a specifying part 361 and the execution part 362. The specifying part 361 detects a user from detection data of the sensor 330 and specifies a user ID of the detected user.

For example, in a case where the sensor 330 is an image sensor, the specifying part 361 detects a user by executing predetermined image recognition processing on image data that is detection data. Then, the specifying part 361 may sequentially compare a feature value of a face registered in the memory 320 with respect to a face image included in the image data, and specify a user ID having similarity equal to or greater than a threshold and corresponding to a maximum feature value as a user ID of a user included in the image data.

For example, in a case where the sensor 330 is a microphone, the specifying part 361 detects voice of a user by executing voice recognition processing on sound data that is detection data. Then, the specifying part 361 may sequentially compare the sound data with a feature value of voice of one or more users registered in advance to specify a user ID.

Although the processing of specifying a user ID using a feature value registered in advance in the memory 320 has been described here, this is just an example. For example, the specifying part 361 may specify a user ID by inputting detection data of the sensor 330 to a user specifying model generated in advance through machine learning such as a neural network.

The execution part 362 acquires a presentation control program corresponding to a user ID specified by the specifying part 361 from the memory 320 and executes the presentation control program. As a result, a content is presented in a display mode and a display method suitable for a physical constitution of a user.

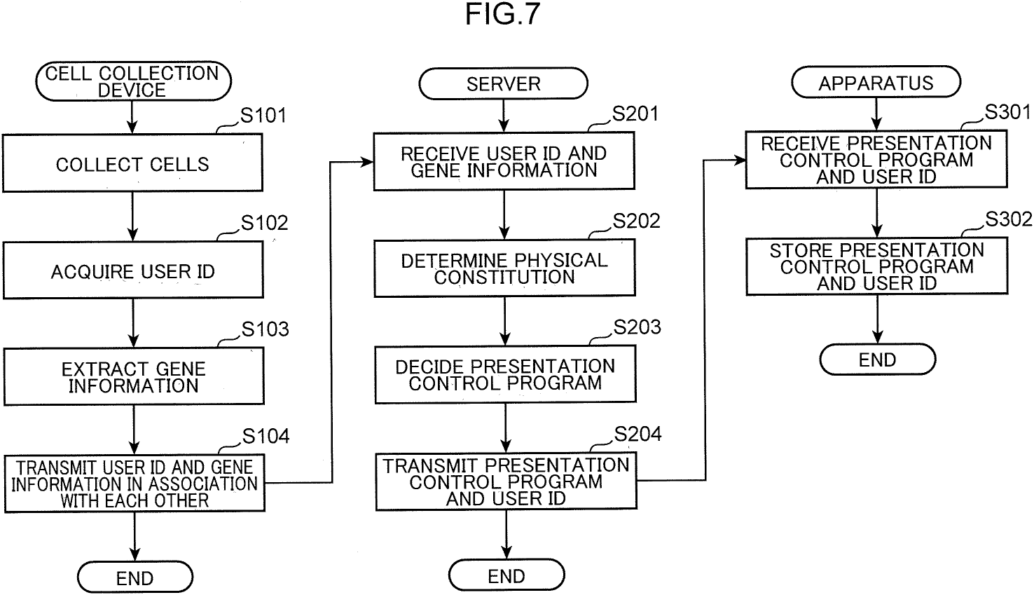

FIG. 7 is a flowchart illustrating an example of processing executed when the control system 1 according to the first embodiment of the present disclosure decides a presentation control program. Note that in this flowchart, for convenience of description, processing for deciding one presentation control program for a certain one apparatus 300 will be described as an example.

In Step S101, the collection unit 130 of the cell collection device 100 collects cells of a user. In Step S102, the user ID acquisition unit 150 acquires a user ID of the user whose cells have been collected. For example, as described above, the user ID acquisition unit 150 may acquire a user ID stored in advance in the memory 140, or may acquire a user ID from the user recognition device 400, or may acquire a user ID input by the user.

In Step S103, the extraction unit 120 extracts gene information from the collected cells. In Step S104, the communication unit 110 transmits the user ID acquired in Step S102 and the gene information extracted in Step S103 to the server 200 in association with each other.

In Step S201, the communication unit 230 of the server 200 receives the user ID and the gene information. In Step S202, the determination part 212 determines a physical constitution of the user who has provided his/her gene information from the gene information received in Step S201. For example, the determination part 212 may specify a user’s physical constitution from SNP and a type of the SNP as described above. Here, as described above, the determination part 212 calculates the evaluation value V1 for evaluating a physical constitution related to attentiveness and the evaluation value V2 for evaluating a physical constitution related to memory.

In Step S203, the decision part 213 decides one presentation control program corresponding to the physical constitution determined in Step S202 from the plurality of presentation control programs predetermined for the apparatus 300. Here, the decision part 213 decides one presentation control program corresponding to a combination of the evaluation value V1 and the evaluation value V2 calculated by the determination part 212.

In Step S204, the communication unit 230 transmits the presentation control program decided in Step S203 and the user ID received in Step S201 to the apparatus 300 in association with each other.

In Step S301, the communication unit 310 of the apparatus 300 receives the presentation control program and the user ID.

In Step S302, the communication unit 310 stores the presentation control program and the user ID received in Step S301 in the memory 320 in association with each other.

The foregoing processing enables the apparatus 300 to acquire a presentation control program according to a physical constitution of an individual user.

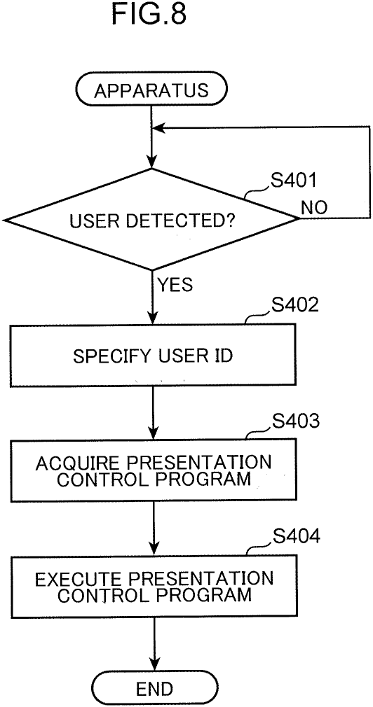

FIG. 8 is a flowchart illustrating an example of processing executed when the apparatus 300 executes a presentation control program. In Step S401, the specifying part 361 detects a user from detection data of the sensor 330. When a user is detected (YES in Step S401), the processing proceeds to Step S402, and when no user is detected (NO in Step S402), the processing returns to Step S401.

In Step S402, the specifying part 361 specifies a user ID of the user detected in Step S401. In Step S403, the execution part 362 acquires a presentation control program corresponding to the user ID specified in Step S402 from the memory 320.

In Step S404, the execution part 362 executes the presentation control program acquired in Step S403.

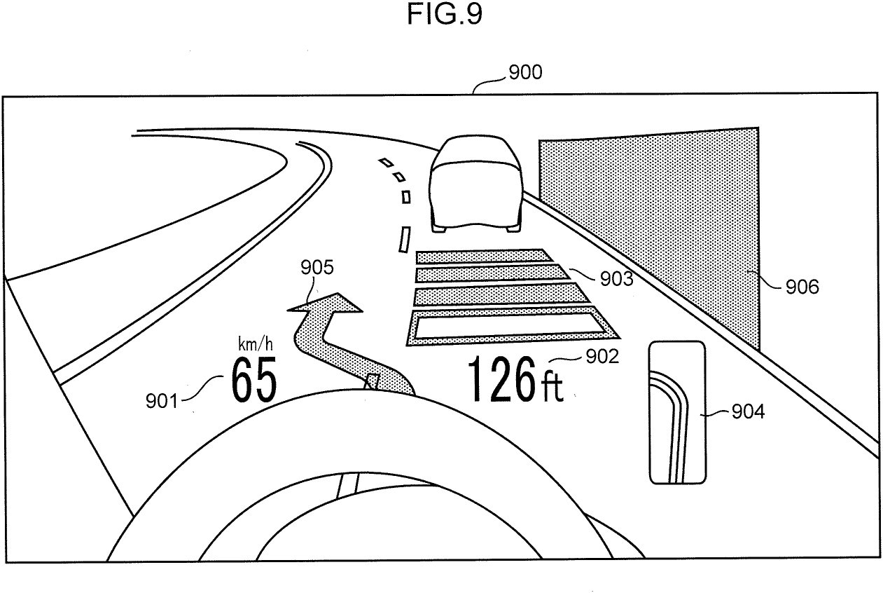

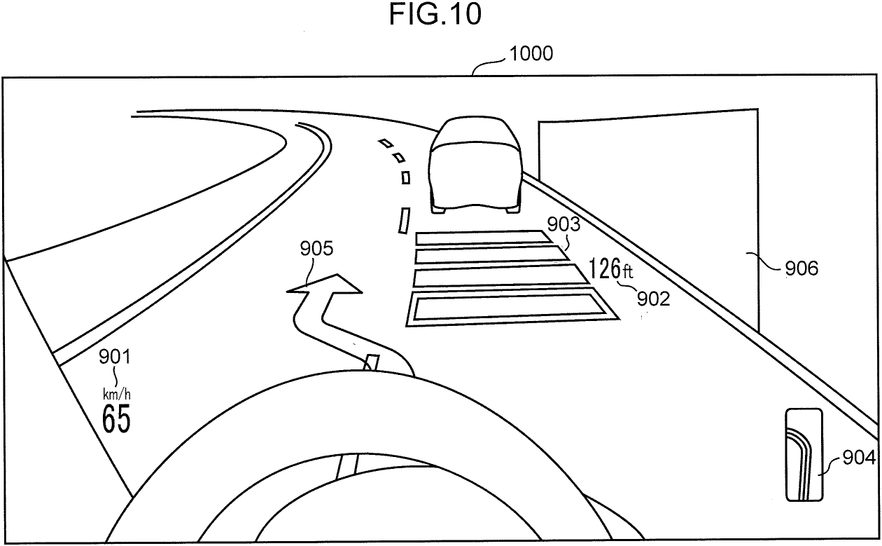

FIG. 9 is a view illustrating an example of an augmented reality screen 900 displayed on the display device 340. FIG. 10 is a view illustrating an example of an augmented reality screen 1000 displayed on the display device 340. The augmented reality screen 900 is a screen displayed for a user having low attentiveness and bad memory (hereinafter, described as attentiveness or the like.). The augmented reality screen 1000 is a screen displayed for a user with high attentiveness or the like. The augmented reality screens 900 and 1000 are images in which contents are superimposed and displayed in a real space. Here, illustrated is an augmented reality screen in which content are superimposed in a real space reflected on a windshield of a vehicle.

In the augmented reality screens 900 and 1000, a scene immediately before entering a left curve is shown. In this scene, the own vehicle is traveling in a right lane of a two-lane road, and the preceding vehicle is traveling ahead in the right lane. The augmented reality screen 900 includes a speed content 901, car distance contents 902 and 903, a road direction content 904, a lane guidance content 905, and a guard content 906.

The speed content 901 is a numerical value image indicating a value of a traveling speed of the own vehicle. The car distance content 902 is a numerical value image indicating a value of a car distance between the own vehicle and the preceding vehicle. The car distance content 903 is configured with a plurality of rectangular object images arranged according to the car distance. The road direction content 904 is an object image representing a shape of a road around a current position when viewed from above. Here, since the left curve is provided ahead, the road direction content 904 includes a road image in which a front end side is curved leftward. The lane guidance content 905 is a bent arrow image that urges a driver to change a lane. Here, an arrow image for guiding lane change from the right lane to a left lane is displayed as the lane guidance content 905. The guard content 906 is a content indicating that the own vehicle is approaching a position before the curve. Here, since the own vehicle is approaching the position before the left curve, an image of a wall rising along the shape of the road is displayed as the guard content 906 at the right side of the road.

These contents are also displayed on the augmented reality screen 1000. The augmented reality screen 900 is a screen displayed for a user with low attentiveness or the like. Therefore, the speed content 901, the car distance content 902, and the road direction content 904 are displayed at a position closer to the center of a user’s field of view in a larger size than in the augmented reality screen 1000. Note that since a field of view of a driver on a windshield can be grasped in advance, the presentation control program need only adjust a display position of a content according to the field of view by designating a display position of the content.

Further, the car distance content 903, the road direction content 904, and the guard content 906 are displayed in a color having a higher contrast with respect to the background than the augmented reality screen 1000. However, this is an example, and all contents included in the augmented reality screen 900 may be displayed in a color having a higher contrast with respect to the background than in the augmented reality screen 1000.

As described above, as compared with the augmented reality screen 1000, in the augmented reality screen 900, the contents are displayed in a large size as a whole near the center of the field of view and with high contrast. As a result, it is possible to cause a user having low attentiveness to recognize a content accurately.

On the other hand, as compared with the augmented reality screen 900, in the augmented reality screen 1000, the contents are displayed in a small size as a whole near an edge of the field of view and with low contrast. As a result, it is possible to cause a user having high attentiveness to recognize a content while suppressing troublesomeness.

Note that although in FIG. 9 and FIG. 10, a content whose display mode and display method (hereinafter, referred to as a display mode and the like) are changed according to attentiveness (hereinafter, the content will be referred to first content) and a content whose display mode and the like are changed according to memory (hereinafter, the content will be referred to as second content) are not distinguished , the first content and the second content may be distinguished.

For example, the car distance content 903, the road direction content 904, the lane guidance content 905, and the guard content 906 may be the first content, and the remaining content may be the second content. In this case, the first content has the display mode or the like changed according to the evaluation value V1, and the second content has the display mode or the like changed according to the evaluation value V2.

Note that since the second content relates to memory, a content including, for example, text which has a large amount of information and is difficult to be intuitively stored, may be adopted. For example, a work list in which works to be performed by a user at the time of returning home are displayed as a list may be adopted as the second content.

Further, an image indicating shaking of a vehicle may be displayed as a content on the augmented reality screens 900 and 1000. In this case, the image indicating shaking of the vehicle may be displayed more times in a case where attentiveness or the like is low than in a case where attentiveness is high. Examples of the content whose number of times of displaying is changed include a content that urges left turn or right turn during navigation to a destination. In a case where attentiveness or the like is low, the number of times of displaying the content for urging left turn or right turn is increased as compared with a case where attentiveness is high.

Further, assuming that the guard content 906 is a content displayed for a certain period of time at timing before entering the curve, the guard content 906 may be displayed for a longer period of time when attentiveness or the like is low than when attentiveness or the like is high. A mode of changing the display time is applicable not only to the guard content 906 but also to any content as long as the content is displayed for a certain period of time at certain timing according to a traveling scene. It is assumed, for example, that a content that urges left turn or right turn during navigation to a destination is displayed. In this case, the content may be displayed for a longer period of time in a case where attentiveness or the like is low than in a case where the attentiveness or the like is high.

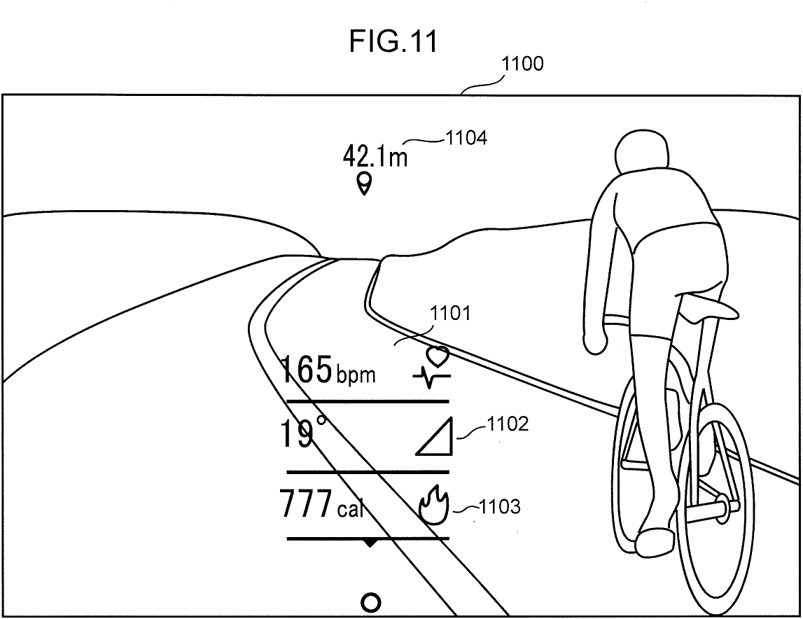

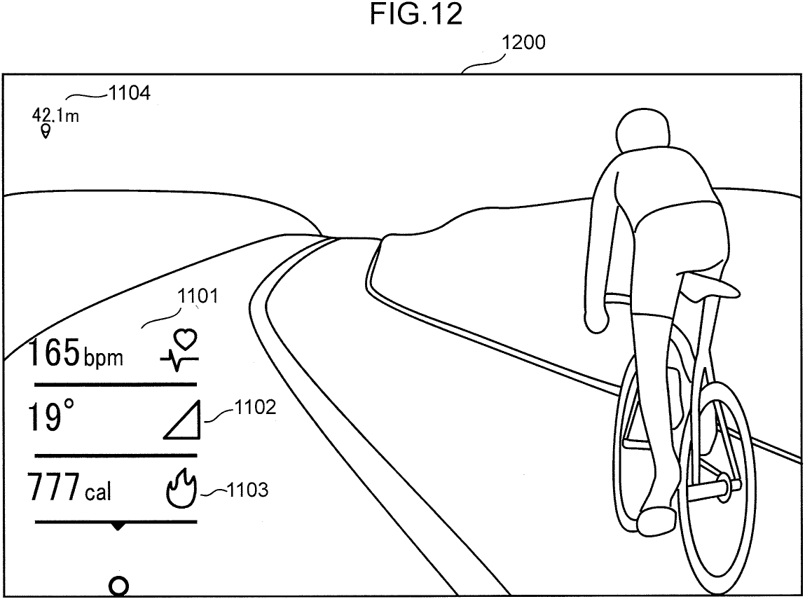

FIG. 11 is a view illustrating an augmented reality screen 1100 according to another example of the present disclosure. FIG. 12 is a view illustrating an augmented reality screen 1200 according to still another example of the present disclosure. The augmented reality screens 1100 and 1200 are screens displayed when the apparatus 300 is smart glasses or smart contact lenses. In the augmented reality screens 1100 and 1200, a scene is illustrated in which a user wearing smart glasses or smart contact lenses is traveling on a bicycle. In this scene, another bicycle is traveling ahead on the right side.

The augmented reality screen 1100 includes a heart rate content 1101, a gradient content 1102, a calorie content 1103, and a remaining distance content 1104. The heart rate content 1101 is a numerical value image indicating a current heart rate of a user. The gradient content 1102 is a numerical value image indicating a gradient angle of a road on which the bicycle is traveling. The calorie content 1103 is a numerical value image indicating consumed calories from start of traveling to the present. The remaining distance content 1104 is a numerical value image indicating a remaining travel distance to a destination. These contents are also displayed on the augmented reality screen 1200. The augmented reality screen 1100 is a screen displayed for a user with low attentiveness or the like. The augmented reality screen 1200 is a screen displayed for a user with high attentiveness or the like. Therefore, in the augmented reality screen 1100, the heart rate content 1101, the gradient content 1102, the calorie content 1103, and the remaining distance content 1104 are displayed at a position closer to the center of a user’s field of view in a larger size than in the augmented reality screen 1200. Note that since a main point of view of a user on smart glasses or smart contact lenses can be grasped in advance, the presentation control program need only adjust a display position of a content according to a field of view by designating a display position of the content.

On the other hand, as compared with the augmented reality screen 1100, in the augmented reality screen 1200, the contents are displayed in a small size as a whole near an edge of the field of view. As a result, it is possible to cause a user having high attentiveness to recognize a content while suppressing troublesomeness.

As described above, according to the present embodiment, since a physical constitution of a user is determined on the basis of gene information, the physical constitution of the user can be accurately determined. Then, one presentation control program is decided from the plurality of presentation control programs for controlling a content to be presented by the apparatus 300 on the basis of the determined physical constitution, and the decided one presentation control program and the user ID are transmitted to the apparatus 300. Therefore, the apparatus 300 can specify a presentation control program corresponding to a user ID. This enables the apparatus 300 to cause an individual user to accurately recognize the content.

### Second Embodiment

In the first embodiment, the server 200 transmits the presentation control program to the apparatus 300 in association with the user ID. In the second embodiment, the server 200 transmits control information for executing a presentation control program to the apparatus 300. Note that in the present embodiment, the same components as those in the first embodiment are denoted by the same reference numerals, and description thereof will be omitted. The block diagram of the first embodiment is used as a block diagram.

Refer to FIG. 3. In the present embodiment, the decision part 213 of the server 200 decides one presentation control program from among a plurality of presentation control programs on the basis of a physical constitution determined by the determination part 212, and transmits control information for executing the decided one presentation control program to the apparatus 300 via the communication unit 230 in association with a user ID. The control information includes, for example, physical constitution information indicating a physical constitution of a user determined by the determination part 212. The physical constitution information includes information obtained by combining the evaluation value V1 regarding attentiveness and the evaluation value V2 regarding memory calculated by the determination part 212.

Refer to FIG. 4. In the present embodiment, the memory 320 of the apparatus 300 stores a plurality of presentation control programs associated with physical constitution information in advance. The memory 320 further stores, in association with each other, the control information (including physical constitution information) and the user ID transmitted from the server 200.

The execution part 362 acquires a presentation control program associated with physical constitution information corresponding to a user ID specified by the specifying part 361 from the memory 320 and executes the acquired presentation control program.

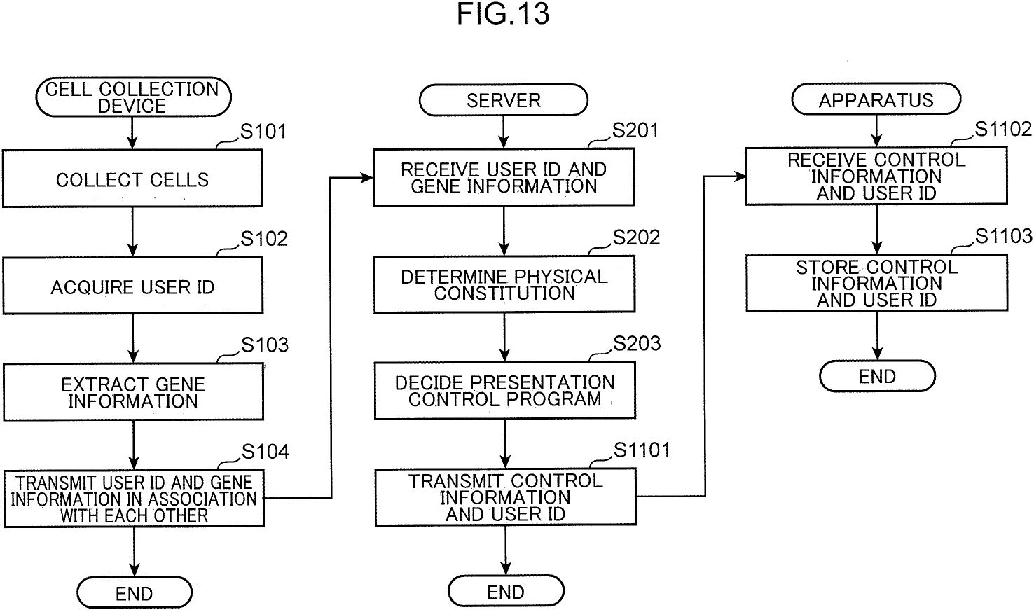

FIG. 13 is a flowchart illustrating an example of processing of the control system 1 according to the second embodiment of the present disclosure. In the present flowchart, the same processing as that in FIG. 7 is denoted by the same processing numeral, and description thereof will be omitted.

In Step S1101 subsequent to Step S203, the communication unit 230 transmits control information for executing the presentation control program decided in Step S203 and the user ID to the apparatus 300.

In Step S1102, the communication unit 310 of the apparatus 300 receives the user ID and the control information. In Step S1103, the communication unit 310 stores the received user ID and control information in the memory 320 in association with each other.

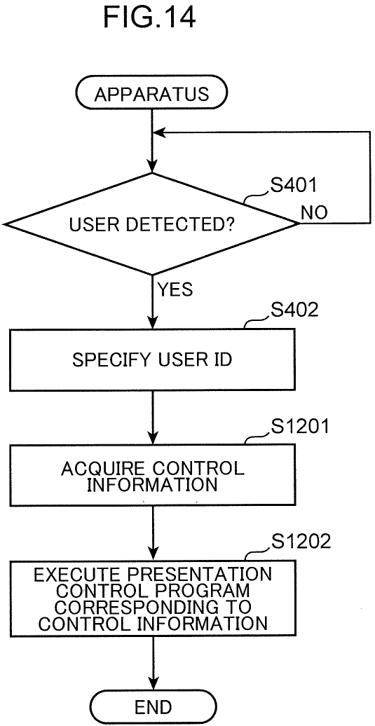

FIG. 14 is a flowchart illustrating an example of processing executed when the apparatus 300 according to the second embodiment of the present disclosure executes the presentation control program. In FIG. 14, the same processing as that in FIG. 8 is denoted by the same processing numeral, and description thereof will be omitted.

In Step S1201 subsequent to Step S402, the execution part 362 acquires control information corresponding to the user ID specified by the specifying part 361 from the memory 320.

In Step S1202, the execution part 362 acquires a presentation control program corresponding to physical constitution information included in the control information from the memory 320 and executes the acquired presentation control program.

As described above, according to the second embodiment, since the plurality of presentation control programs is stored in advance in the apparatus 300 in association with the physical constitution information, the server 200 can cause the apparatus 300 to execute a presentation control program suitable for a physical constitution of a user without transmitting the presentation control program to the apparatus 300.

### Modification

Modifications set forth below can be adopted in the present disclosure.

(1) Although in the first and second embodiments, the apparatus 300 is an onboard apparatus, the present disclosure is not limited thereto. The apparatus 300 may be a work support apparatus that provides various kinds of guidance to workers in a production line of a factory. In this case, for example, a marker image indicating an attachment position of an assembly part, a guidance image indicating a work procedure, a guidance message indicating a work procedure, and an alert image indicating an error in a work procedure can be adopted as the content. Further, in a case where the apparatus 300 is configured with a mobile terminal such as a smartphone, the apparatus 300 may be used for the purpose of navigating a walking route for a pedestrian.

(2) Although in the examples shown in FIG. 9 to FIG. 12, all objects displayed on the augmented reality screen are set as control targets, the present disclosure is not limited thereto, and some objects may be set as the control targets. Further, the number of pieces of contents whose display mode and display method are changed may be increased as attentiveness is decreased.

(3) In the first and second embodiments, the presentation control program associated with a physical constitution having low attentiveness or the like may further cause the speaker 350 to output a sound content in addition to an image content. For example, in a case of notifying a user to turn left or turn right during navigation to a destination, the presentation control program may output a voice message or a sound effect for guiding the user to make a left turn or a right turn in addition to an image of a content displaying the left turn or the right turn for a user having low attentiveness and memory. On the other hand, only the image of the content displaying the left turn or the right turn may be displayed for a user having high attentiveness and memory.

(4) Although in the first and second embodiments, the server 200 acquires the gene information from the cell collection device 100 installed in the house of the user, the present disclosure is not limited thereto. For example, the server 200 may acquire gene information of the user measured by an external organization together with an user ID of the user.

(5) In the present disclosure, the method for determining SNP and a SNP type is not limited to the above-described method, and for example, it is possible to employ a restriction fragment length polymorphism (RFLP) method, a single strand conformation polymorphism (SSCP) method, an SSCP method, a TaqMan PCR method, an SNaP Shot method, an Invader method, a mass spectrometry method, or a method using a DNA microarray. When these methods are adopted, the gene information may include information indicating SNP and a SNP type.

For example, in a case where a method using a DNA macroarray is adopted, the cell collection device 100 is configured with a DNA microarray. In this case, the cell collection device 100 may transmit information on a specific SNP and a type of the SNP out of collected cells to the server 200 as gene information. The determination part 212 of the server 200 may determine a physical constitution of the user from the SNP and the SNP type included in the gene information.

(6) Although the cell collection device 100, the server 200, and the apparatus 300 are separate devices, the present disclosure is not limited thereto, and they may be configured by one device. In this case, “transmit the acquired identification information and control information for causing the apparatus to execute the decided presentation control program in association with each other” means that the identification information and the control information are transmitted in association with each other in the one device.

(7) Although in the first and second embodiments, the description has been made on the premise that one presentation control program is decided from among a plurality of presentation control programs, the present disclosure is not limited thereto.

### INDUSTRIAL APPLICABILITY

According to the present disclosure, since presentation of a content is controlled in accordance with a physical constitution of a user, the present disclosure is particularly useful for an augmented reality apparatus with a limited field of view.

1. A control method of an apparatus that presents a content, comprising: by a computer
acquiring identification information for specifying a user and gene information of the user associated with the identification information;
determining a physical constitution of the user on the basis of the acquired gene information;
deciding a presentation control program for controlling presentation of the content on the basis of the determined physical constitution; and
transmitting, in association with each other, the acquired identification information and control information for causing the apparatus to execute the decided presentation control program.

2. The control method according to claim 1 wherein the physical constitution is a physical constitution related to at least one of attentiveness and memory of a user.

3. The control method according to claim 2, wherein
the determination includes determining whether the attentiveness is low or high on the basis of the gene information, and
the presentation control program decided in a case where the attentiveness is determined to be low is a program that changes at least one of a display mode and a display method of the content so as to make the content be conspicuous as compared with the presentation control program decided in a case where the attentiveness is determined to be high.

4. The control method according to claim 2, wherein
the determination includes determining whether the attentiveness is low or high, and
the presentation control program decided in a case where the attentiveness is determined to be high is a program that changes at least one of a display mode and a display method of the content so as to make the content be less conspicuous as compared with the presentation control program decided in a case where the attentiveness is determined to be low.

5. The control method according to claim 2, wherein
the determination includes determining whether the memory is bad or good, and
the presentation control program decided in a case where the memory is determined to be bad is a program that changes at least one of a display mode and a display method of the content so as to enable the user to better memorize as compared with the presentation control program decided in a case where the memory is determined to be good.

6. The control method according to claim 3, wherein a change of the display mode includes at least one of a change in contrast of the content and a change in size of the content.

7. The control method according to claim 3, wherein a change of the display method includes at least one of a change in a display position of the content, a change in a display time of the content, and a change in the number of times of displaying the content.

8. The control method according to claim 1, wherein the apparatus is an augmented reality apparatus.

9. The control method according to claim 1, wherein the gene information includes information indicating a base sequence of a nucleic acid of the user.

10. The control method according to claim 1, wherein the determination includes detecting a single nucleotide polymorphism of the gene information, and determining the physical constitution on the basis of a detection result.

11. The control method according to claim 1, wherein the control information includes the presentation control program.

12. The control method according to claim 1, wherein the control information includes information for causing the apparatus having a plurality of presentation control programs to execute the decided presentation control program.

13. A control device of an apparatus that presents a content, comprising:
an acquisition part that acquires identification information for specifying a user and gene information of the user associated with the identification information;
a determination part that determines a physical constitution of the user on the basis of the acquired gene information;
a decision part that decides a presentation control program for controlling presentation of the content on the basis of the determined physical constitution; and
a transmission part that transmits, in association with each other, the acquired identification information and control information for causing the apparatus to execute the decided presentation control program.

14. A non-transitory computer readable recording medium storing a program for causing a computer to execute a control method of an apparatus that presents a content, the program causing the computer to:
acquire identification information for specifying a user and gene information of the user associated with the identification information;
determine a physical constitution of the user on the basis of the acquired gene information; decide a presentation control program for controlling presentation of the content on the basis of the determined physical constitution; and
transmit the acquired identification information and control information for causing the apparatus to execute the decided presentation control program in association with each other.

15. An apparatus that presents a content, comprising:
a reception part that receives control information for controlling execution of a presentation control program for controlling presentation of the content, and identification information of a user associated with the control information, the presentation control program being decided on the basis of a physical constitution of the user determined from gene information of the user;
a memory that stores the control information and the identification information in association with each other;
a sensor that detects a surrounding user;
a specifying part that specifies identification information of the surrounding user from detection data of the sensor; and
an execution part that specifies control information corresponding to the specified identification information from the memory and executes a presentation control program corresponding to the specified control information.
